(19) United States
(12) Patent Application Publication
(10) Pub. No.: US 2006/0270638 A1
(43) Pub. Date: Nov. 30, 2006

(54) Delivery of tigecycline in the presence of warfarin

(75) Inventors: John L. Speth, Malvern, PA (US); Dawn M. Harper, Lansdale, PA (US); Donald G. Raible, Devon, PA (US); Gopal Muralidharan, Bangalore (IN)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/429,601

(22) Filed: May 5, 2006

Related U.S. Application Data
(60) Provisional application No. 60/678,204, filed on May 6, 2005.

(51) Int. Cl. A61K 31/65; A61K 31/366

(52) U.S. Cl. 514/153; 514/457

(57) ABSTRACT

The present disclosure is directed to combination therapies of tigecycline and warfarin and methods of administration of tigecycline and warfarin.

“This application claims priority from copending provisional application, application No. 60/678,204 filed May 6, 2005 the entire disclosure of which is hereby incorporated by reference”

In one embodiment, the present disclosure is directed to combination therapies of tigecycline and warfarin and methods of administration of tigecycline and warfarin.

Tigecycline, (9-(t-butyl-glycylamido)-minocycline, TBA-MI NO, (4S,4aS,5aR,12aS)-9-[2-(tert-butylamino)acetamido]-4,7-bis(dimethylamino)-1,4,4a,5,5a,6,11,12a-octahydro-3,10,12,12a-tetrahydroxy-1,11-dioxo-2-naphthacenecarboxamide, is a glycylcycline antibiotic and an analog of the semisynthetic tetracycline, minocycline. Tigecycline is a 9-t-butylglycylamidok derivative of minocycline, formula (I):

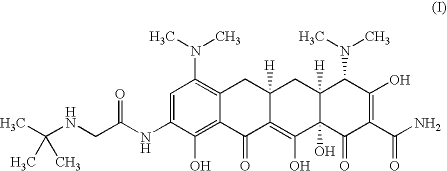

Tigecycline, developed in response to the worldwide threat of emerging resistance to antibiotics, has expanded broad-spectrum antibacterial activity both in vitro and in vivo. Glycylcycline antibiotics, like tetracycline antibiotics, act by inhibiting protein translation in bacteria.

Tigecycline is active against many antibiotic-resistant gram-positive pathogenic bacteria, such as methicillin-resistant Staphylococcus aureus, penicillin-resistant Streptococcus pneumoniae, and vancomycin-resistant enterococci (Eliopoulos, G. M. et al. 1994. Antimicrob Agents Chemother 38:534-41; Fraise, A. P. et al. 1995. Journal of Antimicrobial Chemotherapy. 35:877-81. [erratum appears in J Antimicrob Chemother 1996 May; 37(5):1046]; Garrison, M. W. et al. 2005. Clinical Therapeutics 27:12-22; Goldstein, F. W. et al. 1994. Antimicrobial Agents & Chemotherapy. 38:2218-20; Postier, R. G. et al. 2004. Clin Ther 26:704-714; Weiss, W. J. et al. 1995. Journal of Antimicrobial Chemotherapy. 36:225-30. Tigecycline is also active against bacterial strains carrying the two major forms of tetracycline resistance, efflux and ribosomal protection (Hirata, T. et al. 2004. Antimicrob Agents Chemother. 48:2179-84; Orth, P. et al. 1999. Journal of Molecular Biology 285:455-61; Projan, S. J. 2000. Pharmacotherapy 20:219S-223S; discussion 224S-228S; Schnappinger, D., and W. Hillen. 1996. Archives of Microbiology 165:359-69; Someya, Y. et al. 1995. Antimicrob Agents Chemother. 39:247-249.)

Tigecycline may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, tigecycline may be used to treat or control bacterial infections in warm-blooded animals caused by bacteria having the TetM and TetK resistant determinants. Also, tigecycline may be used to treat bone and joint infections, catheter-related bacteremia, Neutropenia, obstetrics and gynecological infections, or to treat other resistant pathogens, such as VRE, ESBL, enterics, rapid growing mycobacteria, and the like. Hlavaka, et al., U.S. Pat. No. 5,529,990 discloses a method of treating or controlling bacterial infections in warm-blooded animals comprising administering a pharmacologically effective amount of a 7-substituted-9-(substituted amino)-6-demethyl-6-deoxytetracycline, of which tigecycline is a member of the genus described. U.S. Pat. No. 5,529,990 also discloses a method of treating or controlling bacterial infections in warm-blooded animals caused by bacteria having the TetM and TetK resistant determinants comprising administering a pharmacologically effective amount of a 7-substituted-9-(substituted amino)-6-demethyl-6-deoxytetracycline, of which tigecycline is a member of the genus described. U.S. Pat. No. 5,529,990 is incorporated herein by reference in its entirety.

Tigecycline may be prepared by lyophilization and formulated, for example, compounded in the hospital pharmacy, for reconstitution as an IV solution. Tigecycline will frequently be administered simultaneously with other diluents and drugs. Thus, in one embodiment, tigecycline should not interact with or adversely affect the administration of the other drugs when the drugs are given together.

Warfarin sodium is a widely used anticoagulant that acts by inhibiting vitamin K-dependent coagulation factors. In 2004, Americans filled about 16.1 million prescriptions for warfarin. [Top 200 Generic Drugs by Prescription in 2004, http://www.drugtopics.com/drugtopics/article/articleDetail.jsp?id=150069. accessed Mar. 25, 2005]

Chemically, warfarin is 3-(α-acetonylbenzyl)-4-hydroxycoumarin and is a racemic mixture of the R- and S-enantiomers. Crystalline warfarin sodium is an isopropanol clathrate. Its empirical formula is C₁₉H₁₅NaO4, and its structural formula may be represented by the following:

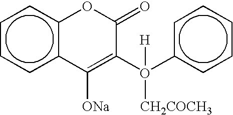

The objective of anticoagulant therapy is to decrease the clotting ability of the blood so that thrombosis is prevented, while avoiding spontaneous bleeding.

Warfarin is, for example, indicated for the prophylaxis and/or treatment of venous thrombosis and its extension, and pulmonary embolism; for the prophylaxis and/or treatment of the thromboembolic complications associated with atrial fibrillation and/or cardiac valve replacement; and to reduce the risk of death, recurrent myocardial infarction, and thromboembolic events such as stroke or systemic embolization after myocardial infarction.

The optimal therapeutic plasma concentration of warfarin shows great interindividual variation, as does the toxic concentration; warfarin has been described as having a narrow therapeutic index. (Ansell, J., and D. Bergqvist. Drugs. 2004, 64 Suppl 1:1-5; Glasheen, J. J. 2005. Southern Medical Journal 98:96-103; Wiftkowsky, A. K. 2004. American Journal of Managed Care 10:S297-306; discussion S312-7.) Any change in this concentration, as a consequence of a pharmacokinetic (PK) interaction with other drugs used concurrently, may therefore lead either to a clinically important reduction in the pharmacological response or to an increase in the incidence of unwanted toxic effects.

The tetracycline class (e.g., tetracycline, chlortetracycline, demeclocycline, minocycline, oxytetracycline, methacycline, and doxycycline), a widely used antibiotic class, is known to have an adverse interaction with warfarin sodium (“warfarin”) generally causing an increase in international normalized ratio (INR) in comparison to administration of warfarin alone. (World Health Organization Technical Report Series. 1983, 687:1-184; Loeliger, E. A. et al. 1985. Thrombosis & Haemostasis 53:148-54; Poller, L. 2004. Journal of Thrombosis & Haemostasis 2:849-60.)

For example, minocycline, of which Tigecycline is an analog, has a demonstrated incompatibility with warfarin. (Caraco, Y., and A. Rubinow. 1992. Annals of Pharmacotherapy 26:1084-6; Ceccaldi, B. et al. 1998. Presse Medicale 27:571; Ciancio, S. G. et al. 1980. Journal of Periodontology 51:530-4; Baciewicz, A. M., and B. S. Bal. Archives of Internal Medicine. 2001, 161:1231; Danos, E. A. 1992. Clinical Pharmacy 11:806-8; Raasch, R. H. 1987. Geriatrics 42:69-74; Westfall, L. K. et al. 1980. American Journal of Hospital Pharmacy 37:1620.

It has suprisingly been found here, however, that tigecycline may be administered with warfarin. Additionally, for example, tigecycline may be administered with warfarin without adjusting the warfarin dose.

Disclosed is a composition comprising at least one glycylcycline, such as tigecycline and warfarin. Another embodiment is a combination therapy comprising administration of at least one glycylcycline and warfarin. Another embodiment is a pharmaceutical composition comprising at least one glycylcycline and warfarin and at least one pharmaceutically acceptable excipient. In one embodiment, tigecycline, as used herein, may be replaced or combined with other glycylcyclines. Also disclosed are methods of using at least one glycylcycline and warfarin.

Another embodiment of the disclosure is a medical apparatus comprising at least two separate compartments, wherein a first compartment comprises at least one glycylcycline and a second compartment comprises warfarin, and wherein the first and second compartments are connected by a line. For example, the first and second compartments may be connected to the same administration set and the contents of the first and second compartments may be mixed prior to administration. By further example, the administration set may contain a Y-site where the contents of the first and second compartments are mixed prior to administration.

### BRIEF DESCRIPTION OF THE DRAWINGS

FIG. 1: Shows mean (SE) R-Warfarin Concentrations in Healthy Subjects Receiving a Single Oral Dose of Warfarin, 25 mg Alone, and With Concurrent Multiple IV Doses of Tigecycline, 50 mg Every 12 Hours

FIG. 2: Shows mean (SE) Plasma S-Warfarin Concentrations in Healthy Subjects Receiving a Single Oral Dose of Warfarin, 25 mg Alone, and With Concurrent Multiple IV Doses of Tigecycline, 50 mg Every 12 Hours

FIG. 3: Shows mean (SE) Prothrombin Time INR in Healthy Subjects Receiving a Single Oral of Warfarin, 25 mg Alone, and With Concurrent Multiple IV Doses of Tigecycline, 50 mg Every 12 Hours

### DEFINITIONS

Throughout the specification and claims, including the detailed description below, the following definitions apply.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

“Glycylcycline” as used herein refers to any glycyl derivative of any tetracycline and includes any salt forms, such as any pharmaceutically acceptable salt, enantiomers and stereoisomers. See Sum P. E. et al. J Med Chem 1994; 37:184-188. Glycylcycline, as used herein, may be formulated according to methods known in the art.

“Tigecycline” as used herein includes tigecycline in free base form and salt forms, such as any pharmaceutically acceptable salt, enantiomers and stereoisomers. Tigecycline, as used herein, may be formulated according to methods known in the art. By way of non-limiting example, tigecycline may optionally be combined with one or more pharmaceutically acceptable excipients, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight. Other formulations are discussed in U.S. Pat. Nos. 5,494,903 and 5,529,990, which are herein incorporated by reference.

“Warfarin” as used herein includes warfarin and its derivates including pharmaceutically acceptable salts, such as sodium salts, and all enantiomers and stereoisomers. Warfarin may also be formulated according to methods known in the art.

“Pharmaceutical composition” as used herein refers to a medicinal composition.

“Pharmaceutically acceptable excipient” as used herein refers to pharmaceutical carriers or vehicles suitable for administration of the compounds provided herein including any such carriers known to those skilled in the art to be suitable for the particular mode of administration. For example, solutions or suspensions used for parenteral, intradermal, subcutaneous, or topical application can include a sterile diluent (e.g., water for injection, saline solution, fixed oil, and the like); a naturally occurring vegetable oil (e.g., sesame oil, coconut oil, peanut oil, cottonseed oil, and the like); a synthetic fatty vehicle (e.g., ethyl oleate, polyethylene glycol, glycerine, propylene glycol, and the like, including other synthetic solvents); antimicrobial agents (e.g., benzyl alcohol, methyl parabens, and the like); antioxidants (e.g., ascorbic acid, sodium bisulfite, and the like); chelating agents (e.g., ethylenediaminetetraacetic acid (EDTA) and the like); buffers (e.g., acetates, citrates, phosphates, and the like); and/or agents for the adjustment of tonicity (e.g., sodium chloride, dextrose, and the like); or mixtures thereof. By further example, where administered intravenously, suitable carriers include physiological saline, phosphate buffered saline (PBS), and solutions containing thickening and solubilizing agents such as glucose, polyethylene glycol, polypropyleneglycol, and the like, and mixtures thereof.

“Administration set” as used herein refers to a device used to administer fluids from a container to a patient's vascular system through a needle or catheter inserted into a vein. The device may, for example, include a needle or catheter, tubing, flow regulators, drip chambers, in-line filters, IV set stopcocks, fluid delivery tubing, infusion pump, connectors between parts of the set, Y-site or side tube with a cap to serve as an injection site, hollow spikes to penetrate and connect the tubing to IV bags or other infusion fluid container.

“Co-administration” as used herein refers to administration of drug A at the same time as drug B, prior to or following the administration of drug B. In one embodiment, the administration is immediately prior or following. In an embodiment of the invention drug A is tigecycline and drug B is warfarin.

“Combination therapy” as used herein refers to a therapy that utilizes co-administration of drug A and drug B. In an embodiment of the invention drug A is tigecycline and drug B is warfarin.

“Administration” as used herein refers to providing a composition orally, parenterally (via intravenous injection (IV), intramuscular injection (IM), depo-IM, subcutaneous injection (SC or SQ), or depo-SQ), sublingually, intranasally (inhalation), intrathecally, topically, or rectally.

“Therapeutically effective amount” as used herein refers to an amount of a therapeutic agent administered to a host to treat or prevent a condition treatable by administration of a composition described in the invention. The amount is the amount sufficient to reduce or lessen at least one symptom of the disease being treated or to reduce or delay onset of one or more clinical markers or symptoms of the disease.

The terms “pharmaceutically acceptable salt” and “salts thereof” refer to acid addition salts or base addition salts of the compounds in the present disclosure. A pharmaceutically acceptable salt is any salt which retains the activity of the parent compound and does not impart any deleterious or undesirable effect on the subject to whom it is administered and in the context in which it is administered. Pharmaceutically acceptable salts include salts of both inorganic and organic acids. Pharmaceutically acceptable salts include acid salts such as acetic, aspartic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Other acceptable salts may be found, for example, in Stahl et al., Pharmaceutical Salts: Properties, Selection, and Use, Wiley-VCH; 1st edition (Jun. 15, 2002).

“Unit dosage form” used herein refers to physically discrete units suitable as unitary dosages, each unit containing a predetermined quantity of active material calculated to produce the desired therapeutic effect.

“Article of manufacture,” “medical apparatus,” and “medical product” as used herein refers to materials useful for prevention or treatment using, for example, tigecycline and warfarin, such as a compartment, container, or vessel with or without a label. The label can be associated with the article of manufacture in a variety of ways including, for example, the label may be on the compartment or the label may be in the compartment as a package insert. Suitable compartments include, for example, blister packs, bottles, bags, vials, syringes, test tubes, and the like. The compartments may be formed from a variety of materials such as glass, metal, plastic, rubber, paper, and the like. The article of manufacture may contain bulk quantities or less of tigecycline. The label on, or associated with, the compartment may provide instructions for the use of tigecycline, instructions for the dosage amount and for the methods of administration including compatibility with warfarin. The article of manufacture may further comprise multiple compartments, also referred to herein as a kit. It may further include other materials desirable from a commercial and user standpoint, including other buffers, diluents, filters, needles, syringes, and/or package inserts with instructions for use.

### DETAILED DESCRIPTION OF THE INVENTION

Tigecycline is an antibiotic that may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, tigecycline may be used to treat or control bacterial infections in warm-blooded animals caused by bacteria having the TetM and TetK resistant determinants. Also, tigecycline may be used to treat bone and joint infections, catheter-related bacteremia, Neutropenia, obstetrics and gynecological infections, or to treat other resistant pathogens, such as VRE, ESBL, enterics, rapid growing mycobacteria, and the like.

Other glycylcycline antibiotics may be used in place of tigecycline or in combination with tigecycline in the practice of the disclosure. Non-limiting examples of other glycylcyclines include (9-(N,N-dimethylglycylamido)-6-demethyl-6-deoxytetracycline), (9-(N,N-dimethylglycylamido)-minocycline), and compounds included in U.S. Pat. No. 5,494,903, which is herein incorporated by reference.

Tigecycline will frequently be administered simultaneously with other diluents and drugs. Thus in one embodiment, the tigecycline should not interact with or adversely effect the dose and administration of the other drugs when the drugs are given together.

It has been discovered that tigecycline may be administered with warfarin and for example, may be administered with warfarin without adjusting the warfarin dose. Specifically, it has been discovered that administration of warfarin with a therapeutic regimen of tigecycline increased the exposure (AUC) of R-warfarin and S-warfarin by 68% and 29%, respectively, but this increase in AUCs did not significantly alter warfarin's anticoagulant profile (measured by INR). No safety-related concerns are identified in healthy subjects after a combined multiple-dose administration of 50 mg IV tigecycline and a single 25-mg oral dose of warfarin. Therefore, no dosage adjustment is warranted when tigecycline is administered with warfarin.

Disclosed is a composition comprising at least one glycylcycline chosen from a compound of the formula 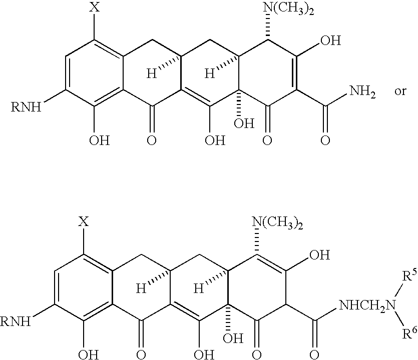
and pharmaceutically acceptable salts thereof, wherein:
X is selected from amino, NR¹R², or halogen; the halogen is selected from bromine, chlorine, fluorine or iodine; R¹ is selected from hydrogen, methyl, ethyl, n-propyl, 1-methylethyl, n-butyl and 1-methylpropyl; R² is selected from methyl, ethyl, n-propyl, 1-methylethyl, n-butyl, 1-methylpropyl, 2-methylpropyl, and 1,1-dimethylethyl such that when X═NR¹R² and R¹=hydrogen,
R²=methyl, ethyl, n-propyl, 1-methylethyl, n-butyl, 1-methylpropyl, 2-methylpropyl or 1,1-dimethylethyl; and when R¹=methyl or ethyl,
R²=methyl, ethyl, n-propyl, 1-methylethyl, n-butyl, 1-methylpropyl or 2-methylpropyl; and when R¹=n-propyl,
R²=n-propyl, 1-methylethyl, n-butyl, 1-methylpropyl or 2-methylpropyl;
and when R¹=1-methylethyl,
R²=n-butyl, 1-methylpropyl or 2-methylpropyl;
and when R¹=n-butyl,
R²=n-butyl, 1-methylpropyl or 2-methylpropyl;
and when R¹=1-methylpropyl,
R²=2-methylpropyl;
R is selected from R⁴(CH₂)ₙCO— or R⁴(CH₂)ₙ SO₂—; and n=0-4;
and when R═R⁴(CH₂)ₙCO— and n-0,
R⁴ is selected from amino; monosubstituted amino selected from straight or branched (C₁-C₆)alkylamino, cyclopropylamino, cyclobutylamino, benzylamino or phenylamino; disubstituted amino selected from dimethylamino, diethylamino, ethyl(1-methylethyl)amino, monomethylbenzylamino, piperidinyl, morpholinyl, 1-imidazolyl, 1-pyrrolyl, 1-(1,2,3-triazolyl) or 4-(1,2,4-triazolyl); a substituted (C₃-C₆)cycloalkyl group with substitution selected from cyano, amino or (C₁-C₃)acyl; a substituted (C₆-C₁₀)aryl group with substitution selected from halo, (C₁-C₄)alkoxy, trihalo (C₁-C₃)-alkyl, nitro, amino, cyano, (C₁-C₄)alkoxycarbonyl (C₁-C₃)alkylamino or carboxy; .-amino-(C₁-C₄)alkyl selected from aminomethyl, .-aminoethyl, .-aminopropyl or .-amino-butyl; carboxy (C₂-C₄)-alkylamino selected from aminoacetic acid, .-aminobutyric acid or .-aminopropionic acid and the optical isomers thereof; (C₇-C₉)aralkylamino; (C₁-C₄)alkoxycarbonylamino substituted (C₁-C₄) alkyl group;
.-hydroxy(C₁-C₃)alkyl selected from hydroxymethyl, .-hydroxyethyl or .-hydroxy-1-methylethyl or .-hydroxypropyl; .-mercapto (C₁-C₃)alkyl selected from mercaptomethyl, .-mercaptoethyl, .-mercapto-1-methylethyl or .-mercaptopropyl; halo-(C₁-C₃)alkyl group; a heterocycle selected from the group consisting of a five membered aromatic or saturated ring with one N, O, S or Se heteroatom optionally having a benzo or pyrido ring fused thereto, a five membered aromatic ring with two N, O, S, or Se heteroatoms optionally having a benzo or pyrido ring fused thereto, a six membered aromatic ring with one to three N, O, S or Se heteroatoms, or a six membered saturated ring with one or two N, O, S or Se heteroatoms and an adjacent appended O heteroatom; acyl or haloacyl group selected from acetyl, propionyl, chloroacetyl, trifluoroacetyl; (C₃-C₆)cycloalcylcarbonyl, (C₆-C₁₀)aroyl selected from benzoyl or naphthoyl; halo substituted (C₆-C₁₀)aroyl; (C₁-C₄) alkylbenzoyl, or (heterocycle)-carbonyl, the heterocycle as defined hereinabove;
(C₁-C₄)alkoxycarbonyl selected from methoxycarbonyl, ethoxycarbonyl, straight or branched propoxylcarbonyl, straight or branched butoxycarbonyl or allyloxycarbonyl; a substituted vinyl group with substitution selected from halogen, halo(C₁-C₃)alkyl, or a substituted (C₆-C₁₀)aryl group with substitution selected from halo, (C₁-C₄)-alkoxy, trihalo(C₁-C₃)alkyl, nitro, amino, cyano, (C₁-C₄)alkoxycarbonyl, (C₁-C₃)alkylamino or carboxy;
(C₁-C₄)alkoxy group; C₆-aryloxy selected from phenoxy or substituted phenoxy with substitution selected from halo, (C₁-C₄) alkyl, nitro, cyano, thiol, amino, carboxy, di(C₁-C₃)alkylamino; (C₇-C₁₀)aralkyloxy; vinyloxy or a substituted vinyloxy group with substitution selected from (C₁-C₄)alkyl, cyano, carboxy, or (C₆-C₁₀)aryl selected from phenyl, .-naphthyl or .-naphthyl; R^{a}R^{b} amino(C₁-C₄)alkoxy group, wherein R^{a}R^{b} is a straight or branched (C₁-C₄)alkyl selected from methyl, ethyl, n-propyl, 1-methylethyl, n-butyl, 1-methylpropyl, or 2-methylpropyl or R^{a}R^{b} is (CH2)m, m=2-6, or (CH₂)₂W(CH₂)₂—wherein W is selected from —N(C₁-C₃)alkyl, O, S, —NH, —NOB and B is selected from hydrogen or (C₁-C₃)alkyl; or R^{a}R^{b} aminoxy group, wherein R^{a}R^{b} is a straight or branched (C₁-C₄)alkyl selected from methyl, ethyl, n-propyl, 1-methylethyl, n-butyl, 1-methylpropyl, 2-methylpropyl, or 1,1-dimethylethyl or R^{a}R^{b} is (CH2)m, m=2-6, or —(CH₂)₂W(CH₂)₂—wherein W is selected from —N(C₁-C₃)alkyl, O, S, —NH, —NOB and B is selected from hydrogen or (C₁-C₃)alkyl;
and when R═R⁴(CH₂)ₙCO— and n-1-4, R⁴ is selected from amino;
a substituted (C₃-C₆)cycloalkyl group with substitution selected from cyano, amino or (C₁-C₃)acyl; a substituted (C₆-C₁₀)-aryl group with substitution selected from halo, (C₁-C₄)-alkoxy, trihalo(C₁-C₃)alkyl, nitro, amino, cyano, (C₁-C₄)alkoxycarbonyl, (C₁-C₃)alkylamino or carboxy; acyloxy or haloacyloxy group selected from acetyl, propionyl, chloroacetyl, trichlorocetyl, (C₃-C₆)cycloalkylcarbonyl, (C₆-C₁₀)aroyl selected from benzoyl or naphthoyl, halo substituted (C₆-C₁₀)aroyl, (C₁-C₄)alkylbenzoyl, or (heterocycle)-carbonyl, the heterocycle as defined hereinabove;
(C₁-C₄)alkoxy; C₆-aryloxy selected from phenoxy or substituted phenoxy with substitution selected from halo, (C₁-C₄)-alkyl, nitro, cyano, thiol, amino, carboxy, di(C₁-C₃)-alkylamino; (C₇-C₁₀)aralkyloxy; (C₁-C₃)alkylthio group selected from methylthio, ethylthio, propylthio or allythio; C₆-arylthio group selected from phenylthio or substituted phenylthio with substitution selected from halo, (C₁-C₄)alkyl, nitro, cyano, thiol, amino, carboxy, di(C₁-C₃)alkylamino; C₆-arylsulfonyl group selected from phenylsulfonyl or substituted phenylsulfonyl with substitution selected from halo, (C₁-C₄)alkoxy, trihalo(C₁-C₃)alkyl, nitro, amino, cyano, (C₁-C₄)alkoxycarbonyl, (C₁-C₃)alkylamino or carboxy; (C7-C8)aralkylthio group; a heterocycle as defined hereinabove; hydroxy; mercapto; mono- or di-straight or branched chain (C₁-C₆)— alkylamino with the alkyl selected from methyl, ethyl, n-propyl, 1-methylethyl, n-butyl, 1-methylpropyl, 2-methylpropyl, 1,1-dimethylethyl, 2-methylbutyl, 1,1-dimethylpropyl, 2,2-dimethylpropyl, 3-methylbutyl, n-hexyl, 1-methylpentyl, 1,1-dimethylbutyl, 2,2-dimethylbutyl, 2-methylpentyl, 1,2-dimethylbutyl, 1,3-dimethylbutyl or 1-methyl-1-ethylpropyl; (C₂C₅)azacycloalkyl group; a carboxy(C₂-C₄)alkylamino group with the carboxy alkyl selected from aminoacetic acid, .-aminopropionic acid, .-aminobutyric acid and the optical isomers thereof; .-hydroxy(C₁-C₃)alkyl selected from hydroxymethyl, .-hydroxyethyl or .-hydroxy-1-methylethyl or .-hydroxypropyl; halo(C₁-C₃)alkyl group; acyl or haloacyl selected from acetyl, propionyl, chloroacetyl, trifluoroacetyl; (C₃-C₆)cycloalkylcarbonyl; (C₆-C₁₀)aroyl selected from benzoyl or naphthoyl; halo substituted (C₆-C₁₀)aroyl; (C₁-C₄)alkylbenzoyl, or (heterocycle)carbonyl, the heterocycle as defined hereinabove;
(C₁-C₄)alkoxycarbonylamino, group selected from tert-butoxycarbonylamino, allyloxycarbonylamino, methoxycarbonylamino, ethoxycarbonylamino or propoxycarbonylamino; (C₁-C₄)alkoxycarbonyl group selected from methoxycarbonyl, ethoxycarbonyl, straight or branched propoxycarbonyl, allyloxycarbonyl or straight or branched butoxycarbonyl; R^{a}R^{b}—amino(C₁-C₄)alkoxy group wherein R^{a}R^{b} is a straight or branched (C₁-C₄)alkyl selected from methyl, ethyl, n-propyl, 1-methylethyl, n-butyl, 1-methylpropyl, or 2-methylpropyl or R^{a}R^{b} is (CH)ₘ m=2-6 or —(CH₂)₂W(CH₂)₂—wherein W is selected from —N(C₁-C₃)-alkyl, O, S, —NH, —NOB, and B is selected from hydrogen or C₁-C₃)alkyl; or R^{a}R^{b} aminoxy group, wherein R^{a}R^{b} is a straight or branched (C₁-C₄)-alkyl selected from methyl, ethyl, n-propyl, 1-methylethyl, n-butyl, 1-methylpropyl, 2-methylpropyl or R^{a}R^{b} is (CH₂)ₘ, m=2-6, or —(CH₂)₂W(CH₂)₂—wherein W is selected from —N(C₁-C₃)-alkyl, O, S, —NH, —NOB and B is selected from hydrogen or (C₀-C₃)alkyl, and when R═R^{4′} (CH₂)ₙ SO₂— and n=0
R^{4′} is selected from amino; monosubstituted amino selected from straight or branched (C₁-C₆)alkylamino, cyclopropylamino, cyclobutylamino, benzylamino or phenylamino; disubstituted amino selected from dimethylamino, diethylamino, ethyl(1-methylethyl)amino, monomethylbenzylamino, piperidinyl, morpholinyl, 1-imidazoyl, 1-pyrrolyl, 1-(1,2,3-triazolyl) or 4-(1,2,4-triazolyl); a substituted (C₃-C₆)cycloalkyl group with substitution selected from cyano, amino or (C₁-C₃)acyl; halo(C₁-C₃)alkyl group; a heterocycle as defined hereinabove;
R^{a}R^{b} amino (C₁-C₄) alkoxy group, wherein R^{a}R^{b} is a straight or branched (C₁-C₄)-alkyl selected from methyl, ethyl, n-propyl, 1-methyl-ethyl, n-butyl, 1-methylpropyl, or 2-methylpropyl or R^{a}R^{b} is (CH2)m, m=2-6, or —(CH₂)₂W—(CH₂)₂—wherein W is selected from —N(C₁-C₃) alkyl, O, S, —NH, —NOB and B is selected from hydrogen or (C₁-C₃)-alkyl; or R^{a}R^{b} aminoxy group, wherein R^{a}R^{b} is a straight or branched (C₁-C₄)alkyl selected from methyl, ethyl, n-propyl, 1-methylethyl, n-butyl, 1-methyl-propyl, or 2-methyl-propyl or R^{a}R^{b} is (CH2)m, m=2-6, or —(CH₂)₂W(CH₂)₂—wherein W is selected from —N(C₁-C₃) alkyl, O, S, —NY, —NOB and B is selected from hydrogen or (C₁-C₃) alkyl; and when R═R^{4′} (CH₂)ₙ SO₂—and n=1-4,
R⁴′ is selected from C₁-C₄)carboxyalkyl; a substituted (C₃-C₆)cyclalkyl group with substitution selected from cyano, amino or (C₁-C₃)-acyl; (C₁-C₄)alkoxy; C₆-aryloxy selected from phenoxy or substituted phenoxy with substitution selected from halo, (C₁-C₃)alkyl, nitro, cyano, thiol, amino, carboxy, di(C₁-C₃) alkylamino; (C₇-C₁₀)aralkyloxy; R^{a}R^{b} amino (C₁-C₄) alkoxy, wherein R^{a}R^{b} is a straight or branched (C₁-C₄)-alkyl selected from methyl, ethyl, n-propyl, 1-methylethyl, n-butyl, 1-methylpropyl, or 2-methylpropyl or R^{a}R^{b} is (CH2)m, m=2-6, or —(CH₂)₂W(CH₂)₂—wherein W is selected from —N(C₁-C₃)alkyl, O, S, —NY, or NOB and B is selected from hydrogen or (C₁-C₃)alkyl; or R^{a}R^{b} aminoxy group, wherein R^{a}R^{b} is a straight or branched (C₁-C₄)alkyl selected from methyl, ethyl, n-propyl, 1-methylethyl, n-butyl, 1-methylpropyl, or 2-methylpropyl or R^{a}R^{b} is (CH2)ₘ, m=2-6, or —(CH₂)₂W(CH₂)₂—wherein W is selected from —N(C₁-C₃)alkyl, O, S, —NH, —NOB and B is selected from hydrogen or (C₁-C₃)alkyl; (C₁-C₃) alkylthio selected from methylthio, ethylthio or n-propylthio; C₆-arylthio selected from phenylthio or substituted phenylthio with substitution selected from halo, (C₁-C₃)alkyl, nitro, cyano, thiol, amino, carboxy, di(C₁-C₃)alkylamino; (C₇-C₈) aralkylthio; a heterocycle as defined hereinabove; hydroxy; mercapto; mono- or di-straight or branched (C₁-C₆)alkyl-amino group the alkyl selected from methyl, ethyl, n-propyl, 1-methylethyl, n-butyl, 1-methylpropyl, 2-methylpropyl, 1,1-dimethylethyl, 2-methylbutyl, 1,1-dimethylpropyl, 2,2-dimethylpropyl, 3-methylbutyl, n-hexyl, 1-methylpentyl, 1,1-dimethylbutyl, 2,2-dimethylbutyl, 2-methylpentyl, 1,2-dimethylbutyl, 1,3-dimethylbutyl or 1-methyl-1-ethylpropyl; halo (C₁-C₃) alkyl; acyl or haloacyl selected from acetyl, propionyl, chloro-acetyl, trifluoroacetyl; (C₃-C₆) cycloalkylcarbonyl; (C₆-C₁₀) aroyl selected from benzoyl or naphthoyl; halo substituted (C₆-C₁₀)aroyl, (C₁-C₄) alkylbenzoyl, or (heterocycle) carbonyl, the heterocycle as defined hereinabove; (C₁-C₄)alkoxycarbonyl selected from methoxycarbonyl, ethoxycarbonyl, straight or branched propoxycarbonyl, allyloxycarbonyl or straight or branched butoxycarbonyl; R⁵ is selected from hydrogen; straight or branched (C₁-C₃) alkyl selected from methyl, ethyl n-propyl or 1-methylethyl; (C₆-C₁₀)aryl selected from phenyl, .-naphthyl or .-naphthyl; (C₇-C₉) aralkyl group; a heterocycle as defined hereinabove; or —(CH₂)ₙCOOR⁷ where n=0-4 and R⁷ is selected from hydrogen; straight or branched (C₁-C₃)alkyl group selected from methyl, ethyl, n-propyl or 1-methylethyl; or (C₆-C₁₀)aryl group selected from phenyl, .-naphthyl or .-naphthyl;
R⁶ is selected from hydrogen; straight or branched (C₁-C₃)alkyl group selected form methyl, ethyl, n-propyl or 1-methylethyl; (C₆-C₁₀)aryl group selected from phenyl, .-naphthyl or .-naphthyl; (C₇-C₉)-aralkyl group; a heterocycle as defined hereinabove; or —CH₂)ₙ(COOR^{7′} where n=0-4 and R^{7′} is selected from hydrogen; straight or branched (C₁-C₃)alkyl selected from methyl, ethyl, n-propyl or 1-methylethyl; or (C₆-C₁₀)aryl selected from phenyl, .-naphthyl or .-naphthyl; with the proviso that R⁵ and R⁶ cannot both be hydrogen; or R⁵ and R⁶ taken together are —(CH₂)₂W(CH₂)₂—, wherein W is selected from (CH2)_{q} and q=0-1, —NH, —N(C₁-C₃)-alkyl, —N(C₁-C₄) alkoxy, oxygen, sulfur or substituted congeners selected from (L or D) proline, ethyl (L or D) prolinate, morpholine, pyrrolidine or piperidine;
   or compounds included in U.S. Pat. No. 5,494,903, which is herein incorporated by reference and at least one warfarin.

In one embodiment of the disclosure, the at least one glycylcycline is tigecycline. For example, a composition comprising at least one glycylcycline chosen from a compound of formula I 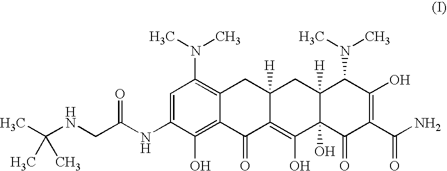
and pharmaceutically acceptable salts thereof, and at least one warfarin. For example, the warfarin may be warfarin sodium. In another embodiment the composition is suitable for parenteral, specifically intravenous, administration.

Another embodiment is a pharmaceutical composition comprising at least one glycylcycline, at least one warfarin and at least one pharmaceutically acceptable excipient. For example, the warfarin may be warfarin sodium. In another embodiment the composition is suitable for parenteral, specifically intravenous, administration.

Another embodiment is a combination therapy comprising administration of at least one glycylcycline and at least one warfarin either sequentially or as a mixture. For example, the warfarin may be warfarin sodium. By further example, the warfarin dose may remain substantially the same as the dose before the combination therapy. In another embodiment the combination therapy is suitable for parenteral, specifically intravenous, administration. By further example, the at least one glycylcycline and the at least one warfarin are administered at substantially the same time or are administered, between twelve hours before and twelve hours after the at least one warfarin is administered, such as for example 10, 8, 6, 4 or 2 hours. For example, the warfarin may be administered at the substantially the same time as tigecycline, with subsequent administration of tigecycline at, for example, 6, 12, 24 or 48 hour intervals thereafter for at least one dosing interval after initial administration. Further, for example, the warfarin may be administered at substantially the same time as tigecycline, with subsequent administration of tigecycline at 12 hour intervals thereafter for 4 days after the initial administration.

Another embodiment of the disclosure is a medical apparatus comprising at least two separate compartments, wherein a first compartment comprises at least one glycylcycline and a second compartment comprises at least one warfarin, and wherein the first and second compartments are connected to an administration set. For example, the first and second compartments may be connected to the same administration set and the contents of the first and second compartments may be mixed prior to administration. By further example, the administration set may contain a Y-site where the contents of the first and second compartments are mixed prior to administration.

Another embodiment is a method for administering at least one glycylcycline and at least one warfarin, comprising administering to a patient in need thereof a therapeutically effective amount of the at least one glycylcycline, and administering to a patient in need thereof a therapeutically effective amount of at least one warfarin. For example, this method and other methods and compositions disclosed may allow the warfarin dose to remain substantially the same as the dose before administration with at least one glycylcycline. For example, the warfarin may be warfarin sodium. In another embodiment the composition is suitable for parenteral, specifically intravenous, administration. By further example, the at least one glycylcycline and the at least one warfarin are administered at substantially the same time or are administered between twelve hours before and twelve hours after the at least one warfarin is administered, such as for example 10, 8, 6, 4 or 2 hours. For example, the warfarin may be administered at the substantially the same time as tigecycline, with subsequent administration of tigecycline at, for example, 6, 12, 24 or 48 hour intervals thereafter for at least one dosing interval after initial administration. Further, for example, the warfarin may be administered at substantially the same time as tigecycline, with subsequent administration of tigecycline at 12 hour intervals thereafter for 4 days after the initial administration.

A further embodiment is a method of administering an antibiotic comprising administering to a patient in need thereof a therapeutically effective amount of at least one glycylcycline, and administering to a patient in need thereof a therapeutically effective amount of at least one warfarin. By further example, the at least one glycylcycline and the at least one warfarin are administered at substantially the same time or are administered between twelve hours before and twelve hours after the at least one warfarin is administered, such as for example 10, 8, 6, 4 or 2 hours. For example, the warfarin may be administered at the substantially the same time as tigecycline, with subsequent administration of tigecycline at, for example, 6, 12, 24 or 48 hour intervals thereafter for at least one dosing interval after initial administration. Further, for example, the warfarin may be administered at substantially the same time as tigecycline, with subsequent administration of tigecycline at 12 hour intervals thereafter for 4 days after the initial administration.

Another embodiment is a method of treating bacterial infections, such as complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) comprising administering to a patient in need thereof a therapeutically effective amount of at least one glycylcycline, and administering to a patient in need thereof a therapeutically effective amount of at least one warfarin. By further example, in the methods disclosed, the at least one glycylcycline and the at least one warfarin are administered at substantially the same time or are administered between twelve hours before and twelve hours after the at least one warfarin is administered, such as for example 10, 8, 6, 4 or 2 hours. For example, the warfarin may be administered at the substantially the same time as tigecycline, with subsequent administration of tigecycline at, for example, 6, 12, 24 or 48 hour intervals thereafter for at least one dosing interval after initial administration. Further, for example, the warfarin may be administered at substantially the same time as tigecycline, with subsequent administration of tigecycline at 12 hour intervals thereafter for 4 days after the initial administration.

Another embodiment is a method of administering an antibiotic to a patient receiving warfarin comprising administering to a patient in need thereof a therapeutically effective amount of at least one glycylcycline. Another embodiment is a method of administering at least one glycylcycline to a patient receiving warfarin comprising administering to a patient in need thereof a therapeutically effective amount of at least one glycylcycline.

Another embodiment is a method of using at least one glycylcycline in the treatment of a bacterial infection, such as those disclosed herein comprising providing a patient with a therapeutically effective amount of at least one glycylcycline and informing the patient and/or administering medical personnel that the warfarin dose may remain substantially the same as the dose before administration with at least one glycylcycline.

Another embodiment is a composition comprising at least one glycylcycline, such as a pharmaceutical composition, or any composition disclosed herein, comprising packaging the composition with information that the at least one glycylcycline, such as tigecycline, may be administered with warfarin. For example, the packaging may explain that the warfarin dose may remain substantially the same as the dose before administration with at least one glycylcycline. Also disclosed in a method of supplying such a composition to medical personal or a patient in need thereof.

Another embodiment is a method or composition disclosed herein that further comprises a container or compartment with printed labeling advising that the at least one glycylcycline, such as tigecycline, may be administered with warfarin. For example, advising that the warfarin dose may remain substantially the same as the dose before administration with at least one glycylcycline. For example, a composition or method disclosed herein may further provide information that when a therapeutically effective amount of at least one glycylcycline is administered with warfarin, the warfarin dose may remain substantially the same as the dose before administration with at least one glycylcycline. By further example, a composition or method disclosed herein may further provide information that when a therapeutically effective amount of at least one glycylcycline is administered with warfarin, the glycylcycline dose may remain substantially the same as the dose before administration with at least one warfarin.

One embodiment is packaging a composition comprising at least one glycylcycline with information at the at least one glycylcycline may be administered with warfarin, wherein, for example, the warfarin dose may remain substantially the same as the dose before administration with at least one glycylcycline, and methods of using such a composition.

In another embodiment, at least one glycylcycline may be provided in kits, optionally including component parts that can be assembled for use. For example, at least one glycylcycline in lyophilized form and a suitable diluent may be provided as separated components for combination prior to use. A kit may include a plurality of compartments, each compartment holding at least one unit dose of at least one glycylcycline. The compartments are preferably adapted for the desired mode of administration, including, for example, pill, tablet, capsule, powder, gel or gel capsule, sustained-release capsule, or elixir form, and/or combinations thereof, and the like for oral administration, depot products, pre-filled syringes, ampoules, vials, and the like for parenteral administration, and patches, medipads, creams, and the like for topical administration.

For example, a kit may comprise (a) at least one dosage form of at least one glycylcycline; (b) at least one compartment in which at least one glycylcycline is stored; and may also comprise (c) a package insert comprising: i) information regarding the dosage amount and duration of exposure of a dosage form of at least one glycylcycline and/or ii) providing that the dosage form of at least one glycylcycline may be administered with warfarin wherein the warfarin dose may remain substantially the same as the dose before administration with at least one glycylcycline.

In another embodiment, an article of manufacture may comprise a compartment holding at least one glycylcycline in combination with printed labeling instructions providing a discussion that indicates the compatibility of warfarin and the at least one glycylcycline, for example, as opposed to other tetracyclines. The labeling instructions may be consistent, for example, with the methods of treatment as described hereinbefore. The labeling may be associated with the compartment by any means that maintain a physical proximity of the two, by way of non-limiting example, they may both be contained in a packaging material such as a box or plastic shrink wrap or may be associated with the instructions being bonded to the compartment such as with glue that does not obscure the labeling instructions or other bonding or holding means.

For example, an article of manufacture may comprise (a) a dosage form of at least one glycylcycline, (b) a package insert or printed labeling providing that a dosage form of the at least one glycylcycline may be co-administered with warfarin wherein the warfarin dose may remain substantially the same as the dose before administration with at least one glycylcycline; and (c) at least one compartment in which the at least one glycylcycline is stored.

As a non-limiting example, an effective amount of tigecycline ranges from 0.5 mg/kg of body weight to 100.0 mg/kg of body weight, for example, from 0.5 to 15 mg/kg of body weight, by further example, from 0.5 to 1 mg/kg of body weight, may be administered from one to five times per day. For example, tigecycline may be administered as a 100 mg loading dose followed by subsequent administration of 50 mg both administered to the patient via infusion over a 30-60 minute period. In the above example, the loading dose of 100 mg may be prepared by adding two vials of reconstituted tigecycline to 100 mL Normal Saline or dextrose 5% in water (“D5W”) intravenous compartments resulting in a final concentration of 1 mg/mL, whereas the subsequent dose of 50 mg may be prepared by adding one vial of reconstituted tigecycline to 100 mL Normal Saline or D5W intravenous compartments resulting in a final concentration of 0.5 mg/mL. It will be understood, however, that the specific dose level and frequency of dosage for any particular patient may be varied and will depend on a variety of factors including age, body weight, general health, sex, diet, the severity of the condition being treated, and the like.

The reconstitution of tigecycline would be understood by one skilled in the art, for example, by following instructions included by the manufacturer or distributor or by using common medical procedures, which are inclusive of using only a sterile acceptable reconstitution medium and sterile administration compartments as described herein and in the tigecycline product label, to reconstitute and administer the lyophilized tigecycline free base supplied by the manufacturer.

The dosage and administration of warfarin are related to the patient's response to the drug as measured by PT/INR. The prothrombin time (PT) reflects the depression of vitamin K dependent clotting Factors VII, X and II and represents the degree of anticoagulation. A system of standardizing the PT in oral anticoagulant control was introduced by the World Health Organization in 1983. World Health Organization Technical Report Series. 1983, 687:1-184.

It is based upon the determination of an International Normalized Ratio (INR) which provides a common basis for communication of PT results and interpretations of therapeutic ranges. The INR system of reporting is based on a logarithmic relationship between the PT ratios of the test and reference preparation. The INR is the PT ratio that would be obtained if the International Reference Preparation (IRP), which has an ISI of 1.0, was used to perform the test. Early clinical studies of oral anticoagulants, which formed the basis for recommended therapeutic ranges of 1.5 to 2.5 times control mean normal PT, used sensitive human brain thromboplastin. The INR can be calculated as: INR=(observed PT ratio)^{ISI} where the ISI (International Sensitivity Index) is the correction factor in the equation that relates the PT ratio of the local reagent to the reference preparation and is a measure of the sensitivity of a given thromboplastin to reduction of vitamin K-dependent coagulation factors; the lower the ISI, the more “sensitive” the reagent and the closer the derived INR will be to the observed PT ratio. The most commonly American College of Chest Physicians (ACCP)-reported INR is 2.0-3.0. Geerts, W. H. et al. 2004. Chest 126:338S-400S; Monagle, P. et al. 2004. Chest 126:645S-687S; Rockson, S. G., and G. W. Albers. 2004. Journal of the American College of Cardiology 43:929-35; Yim, J. M. et al. 1996. Annals of Pharmacotherapy 30:1390-5.

Other than in the examples, and where otherwise indicated, all numbers used in the specification and claims are to be understood as modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following example is intended to illustrate the invention in a non-limiting manner.

### EXAMPLE 1

### Pharmacokinetic Study of the Potential Drug Interaction Between Tigecycline and Warfarin

This is an open-label, nonrandomized, inpatient/outpatient, 2-period, 2-treatment study performed with healthy subjects at a single investigational site.

Dose and Administration:

Sterile tigecycline powder for injection is supplied in 5-mL, flint glass vials, each containing lyophilized free base equivalent to 50 mg of tigecycline without additives or preservatives. The contents of the vials are reconstituted with sterile normal saline (0.9% NaCl Injection, USP). Warfarin sodium is supplied by as Coumadin® 10-mg and 5-mg tablets.

All subjects received a 25-mg oral dose of warfarin on study day 1 of period 1. They are discharged from the study site on study day 8 to begin a 5-day washout interval, during which no test article is given. On study days 1 through 8 of period 2, subjects received a 100-mg loading dose of tigecycline, followed by 15 doses of 50 mg of IV tigecycline, 1 dose every 12 hours. All tigecycline infusions are administered in a volume of 100 mL of normal saline over 30 minutes at 200 mL/h via an infusion pump. On study day 5 of period 2, subjects receive a 25-mg oral dose of warfarin immediately before the administration of tigecycline. All subjects consume a medium-fat meal approximately 1 hour before administration of the test item(s).

Pharmacokinetic, Pharmacodynamic, and Statistical Methods:

Blood samples are collected on day 1 of period 2 before tigecycline infusion and on days 4 and 5 at time 0 (predose), 0.5 (end of infusion), 1, 1.5, 2, 3, 4, 6, 8, and 12 hours after the start of the tigecycline infusion for determination of tigecycline serum concentrations. Additionally, blood samples are collected at time 0 (predose), 0.5, 1, 2, 3, 4, 6, 8, 12, 24, 36, 48, 72, 96, 120, 144, and 168 hours after the administration of warfarin on day 1 of period 1 and day 5 of period 2 for determination of R-warfarin and S-warfarin plasma concentrations and for determination of prothrombin time (PT) and international normalized ratio (INR). Standard noncompartmental PK methods are used to analyze the tigecycline serum concentration data and the R-warfarin and S-warfarin plasma concentration data. A 2-factor analysis of variance (ANOVA) is used to compare the tigecycline serum concentrations, R-warfarin and S-warfarin plasma concentrations, and PK parameters between the monotherapy and combination therapy, and a 2-factor analysis of covariance (ANCOVA) is used to compare the PT and INR between the warfarin monotherapy and the combination therapy.

Blood Sample Collection and Processing:

Venous blood samples (3 mL) for determination of warfarin concentration in serum are collected on study days 1 through 8 of period 1 and study days 5 through 12 of period 2 at the following times: within 2 hours before sodium warfarin dose administration, and at hours 0.5, 1, 2, 3, 4, 6, 8, 12, 24, 36, 48, 72, 96, 120, 144, and 168 after sodium warfarin dose administration.

Venous blood samples (7 mL) for tigecycline PK determination in serum are collected during period 2 of the study at the following times: study day 1 (predose—collected 2 hours before tigecycline administration), at hour 12 (before the evening dose of tigecycline), and on study days 4 and 5 immediately before tigecycline morning administration and at 0.5, 1, 1.5, 2, 3, 4, 6, 8, and 12 hours after the 8 AM dose.

Tigecycline blood samples are collected from an indwelling catheter or by direct venipuncture. If a catheter is used for blood collection, then approximately 0.5 mL of blood is to be discarded before collecting the sample in a tube that did not contain any anticoagulant.

The total amount of blood collected from each subject is approximately 640 mL. Blood for warfarin analysis is gently mixed with the warfarin in the collection tube and the tube is placed on ice immediately. The tubes are centrifuged within 15 minutes of collection at approximately 1000 g for about 10 minutes. The separated plasma is then transferred to a watertight, labeled polypropylene tube and stored frozen in an upright position at −70° C. or colder until shipped.

Blood tubes for tigecycline analysis are placed immediately on ice until a clot forms. The tubes are centrifuged at approximately 1000 g for about 10 minutes in a refrigerated centrifuge. The serum is collected and transferred into a labeled polypropylene tube and stored in an upright position at −70° C. or colder until shipped.

Sample Analysis:

Concentrations of tigecycline in serum is determined by sensitive and specific liquid chromatography methods with tandem mass spectrometer detection (LC/MS/MS), and concentrations of R(+)-warfarin and S(−)-warfarin in plasma is determined by a separate LC/MS/MS procedure. The performance of the tigecycline and warfarin assays during the analysis of the serum and plasma samples from this study is summarized in Tables 1, 2 and 3.

TABLE 1
Assay Range and Sensitivity

| | Tigecycline/ | R-warfarin/ | S-warfarin/ |
|---|---|---|---|
| Standard | Serum | Plasma | Plasma |
| Curve | (ng/mL) | (ng/mL) | (ng/mL) |
| Linear range | 10.0-2000 | 5.0-1000 | 5.0-1000 |
| Sensitivity | 10.0 | 5.0 | 5.0 |

TABLE 2
Summary of Assay Performance for Tigecycline Serum Assays

| | Low QC (25 ng/mL) | | | Middle QC (200 ng/mL) | | | High QC (1500 ng/mL) | | |
|---|---|---|---|---|---|---|---|---|---|
| Analyte | Mean | CV % | Bias % | Mean | CV % | Bias % | Mean | CV % | Bias % |
| Tigecycline | 27.1 | 8.56 | +8.55 | 210.4 | 2.47 | +5.23 | 1585.6 | 1.62 | +5.71 |

TABLE 3
Summary of Assay Performance for Warfarin Plasma Assays

| | Low QC | | | Middle QC | | | High QC | | |
|---|---|---|---|---|---|---|---|---|---|
| | (10.0 μg/mL) | | | (80.0 μg/mL) | | | (800 μg/mL) | | |
| Analyte | Mean | CV % | Bias % | Mean | CV % | Bias % | Mean | CV % | Bias % |
| R-warfarin | 9.73 | 2.86 | −2.75 | 78.6 | 2.26 | −1.69 | 797 | 2.87 | −0.33 |
| S-warfarin | 9.69 | 3.12 | −3.08 | 78.4 | 2.02 | −1.94 | 798 | 3.32 | −0.19 |

Pharmacokinetic Analysis

The steady-state tigecycline serum concentration data for each subject is analyzed by using empirical, model-independent pharmacokinetic methods. Thus, the peak concentration (Cₘₐₓ) and the time to peak concentration (tₘₐₓ) are taken directly from the observed data. The area under the concentration-time curve over 1 dosage interval (AUC₀₋₁₂ₕ) is calculated by using the log-trapezoidal rule for decreasing concentrations and the linear-trapezoidal rule for increasing concentrations. Systemic clearance (Cl) is calculated as dose/AUC, and it is presented both corrected and uncorrected for body weight.

The single-dose R-warfarin and S-warfarin plasma concentration data for each subject also are analyzed by using empirical, model-independent pharmacokinetic methods. The apparent terminal-phase disposition rate constant (λ_{z}) is estimated by a log-linear regression of the last 2 to 5 observed serum concentrations that are determined to be in the log-linear elimination phase by visual inspection. The apparent terminal-phase disposition half-life (t_{1/2}) is calculated as t_{1/2}=0.693/λ_{z}. The area under the concentration-time curve (AUC_{T}) to the last observable concentration (C_{T}) at time T is calculated by using the log-trapezoidal rule for decreasing concentrations and the linear-trapezoidal rule for increasing concentrations, and the total AUC is estimated by AUC=AUC_{T}+C_{T}/λ_{z}. The oral-dose clearance (Cl/F) is calculated as dose/AUC, and the apparent terminal-phase volume of distribution (V_{z}/F) is estimated as V_{z}/F═[Cl/F]/λ_{z}.

Pharmacodynamic Analysis

To evaluate the clinical relevance of any potential effect of tigecycline on the plasma concentrations of R-warfarin and S-warfarin, the prothrombin time (expressed in seconds and INR) is measured at predose and at 0.5, 1, 2, 3, 4, 6, 8, 12, 24, 36, 48, 72, 96, 120, 144, and 168 hours after each single-dose warfarin administration. The peak INR (INRₘₐₓ) and the time to peak INR are taken directly from the observed data. The area under the INR vs time curve over the 168-hour observation interval is calculated using the linear trapezoidal rule.

Pharmacokinetics Results:

The following table summarizes the PK profile of tigecycline.

Tigecycline Steady-State Pharmacokinetic Parameters (Mean ± SD)

| | Cₘₐₓ | tₘₐₓ | C_{12 h} | AUC_{0-12 h} | CI |
|---|---|---|---|---|---|
| Treatment | (ng/mL) | (h) | (ng/mL) | (ng · h/mL) | (L/h/kg) |
| Tigecycline | 886 ± 202 | 0.50 ± 0.00 | 109 ± 28 | 2097 ± 514 | 0.310 ± 0.072 |
| Tigecycline + warfarin | 898 ± 192 | 0.50 ± 0.00 | 114 ± 32 | 2192 ± 494 | 0.296 ± 0.057 |
| p-value^{a} | 0.646 | — | 0.255 | 0.004 | 0.004 |
| Geo. Mean | 102% | — | 108% | 107% | 93% |
| Ratio^{b} | | | | | |
| 90% CL^{b} | 94-112% | — | 96-123% | 104-111% | 90-96% |

Abbreviations:
AUC_{0-12 h} = area under the concentration time curve during the dose interval 0 to 12 hours;
C_{12 h} = concentration at hour 12;
Cₘₐₓ = peak concentration;
CL = confidence limits;
CI = intravenous clearance;
tₘₐₓ = time to peak concentration
^{a}Treatment p-value from a 2-factor ANOVA of log-transformed data.
^{b}Geometric mean ratio and 90% confidence limits.

Following coadministration of warfarin, the mean steady-state tigecycline systemic clearance and exposure (Cₘₐₓ, C₁₂ₕ, and AUC) are within 10% of the mean values for tigecycline monotherapy. Additionally, the 90% confidence limits of the geometric mean parameter ratio are all within the strict bioequivalence interval criteria of 80% to 125%. Thus, coadministration of warfarin does not alter the PK profile of tigecycline.

The following table summarizes the pharmacokinetic profile of R-warfarin and S-warfarin.

R-Warfarin and S-Warfarin Single-Dose Pharmacokinetic Parameters (Mean ± SD)

| | R-warfarin | | | S-warfarin | | |
|---|---|---|---|---|---|---|
| | Cₘₐₓ | AUC | Cl/F | Cₘₐₓ | AUC | Cl/F |
| Treatment | (ng/mL) | (μg · h/mL) | (mL/h/kg) | (ng/mL) | (μg · h/mL) | (mL/h/kg) |
| Warfarin | 1158 ± 259 | 73 ± 18 | 4.3 ± 0.8 | 1104 ± 282 | 47 ± 18 | 7.0 ± 1.9 |
| Tigecycline + | 1584 ± 259 | 122 ± 28 | 2.5 ± 0.5 | 1555 ± 236 | 59 ± 17 | 5.4 ± 1.2 |
| Warfarin | | | | | | |
| p-value^{a} | 0.001 | 0.001 | 0.001 | 0.001 | 0.001 | 0.001 |
| Geo. Mean | 138% | 168% | 60% | 143% | 129% | 77% |
| Ratio^{b} | | | | | | |
| 90% CL^{b} | 126-151% | 150-187% | 54-67% | 129-158% | 119-140% | 71-84% |

Abbreviations:
AUC = area under the time concentration curve;
Cl/F = oral dose clearance;
Cₘₐₓ = peak concentration.
^{a}Treatment p-value from a 2-factor ANOVA of log-transformed data.
^{b}Geometric mean ratio and 90% confidence limits.

Coadministration of the therapeutic regimen of tigecycline significantly decreased the oral-dose clearance of both R-warfarin and S-warfarin, thereby increasing the Cₘₐₓ and AUC of both compounds.

Pharmacodynamic Results:

The following table summarizes the INR pharmacodynamic profile of warfarin.

INR Pharmacodynamic Parameters (Mean ± SD)

| Treatment | INRₘₐₓ | tₘₐₓ (h) | AUC_{0-168 h} (h) |
|---|---|---|---|
| Warfarin | 1.8 ± 0.3 | 30 ± 9 | 216 ± 14 |
| Tigecycline + Warfarin | 1.6 ± 0.2 | 39 ± 6 | 220 ± 13 |
| P-value^{a} | 0.036 | 0.068 | 0.564 |
| Geo. Mean Ratio^{b} | 90% | 136% | 102% |
| 90% CL^{b} | 84-107% | 104-179% | 97-107% |

Abbreviations:
AUC_{0-168 h} = area under the time concentration curve for the interval 0 to 168 hours;
INR = international normalized ratio;
tₘₐₓ = time to maximum INR value.
^{a}Treatment p-value from a 2-factor ANOVA of log-transformed data.
^{b}Geometric mean ratio and 90% confidence limits.

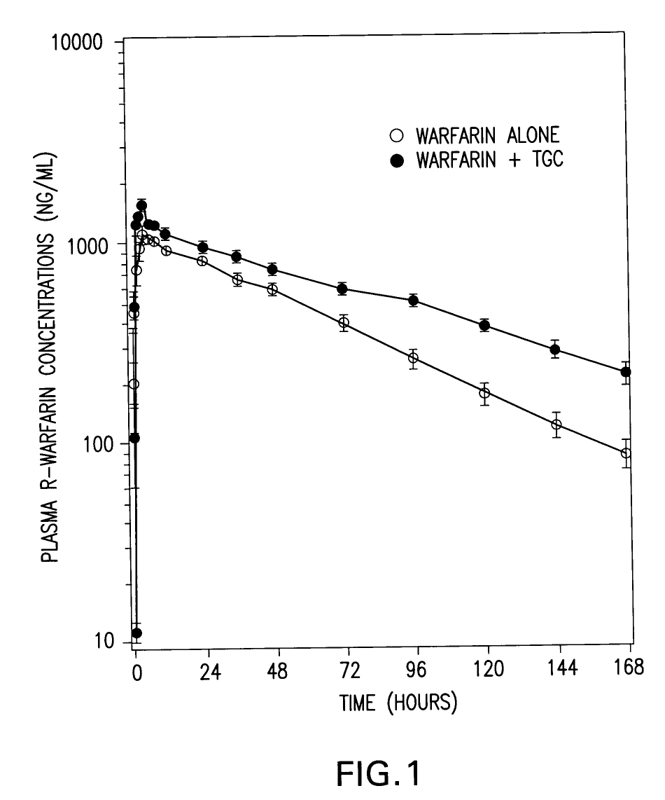

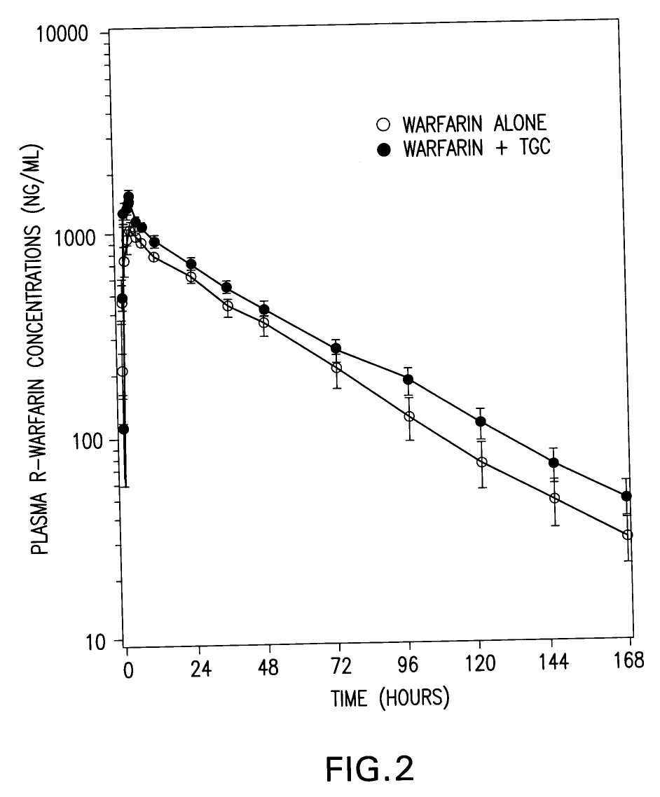

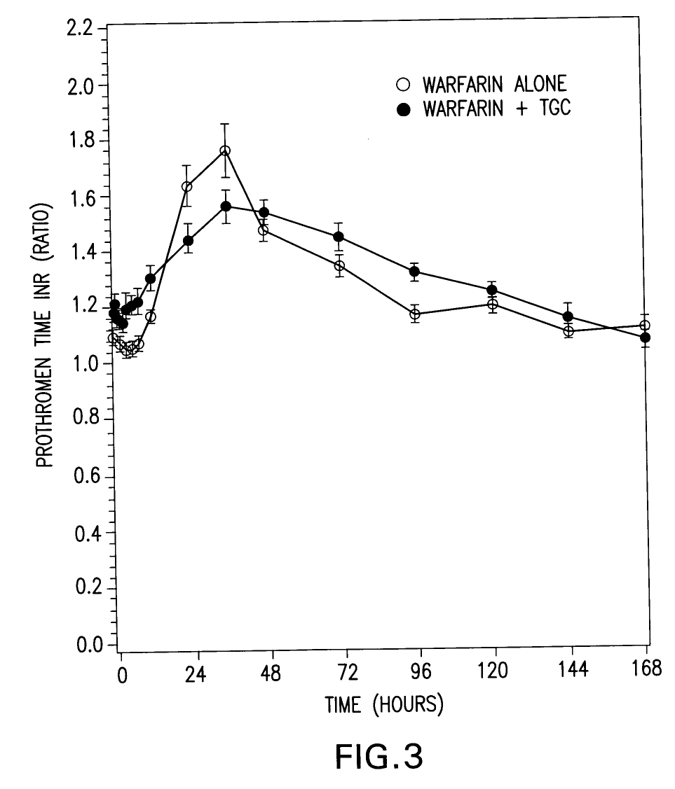

Although the R-warfarin and S-warfarin plasma concentrations are higher following tigecycline coadministration (See FIG. 1: Mean (SE) R-Warfarin Concentrations in Healthy Subjects Receiving a Single Oral Dose of Warfarin 25 mg Alone and With Concurrent Multiple IV Doses of Tigecycline 50 mg Every 12 Hours and FIG. 2: Mean (SE) Plasma S-Warfarin Concentrations in Healthy Subjects Receiving a Single Oral Dose of Warfarin 25 mg Alone and With Concurrent Multiple IV Doses of Tigecycline 50 mg Every 12 Hours), the mean INRₘₐₓ values are 10% lower following administration of the combination treatment, and the mean INR AUC₀₋₁₆₈ₕ values are unchanged (See FIG. 3: Mean (SE) Prothrombin Time INR in Healthy Subjects Receiving a Single Oral of Warfarin 25 mg Alone and With Concurrent Multiple IV Doses of Tigecycline 50 mg Every 12 Hours.) Additionally, the INRₘₐₓ and INR AUC₀₋₁₆₈ₕ meets the strict bioequivalence criteria for comparisons between the warfarin monotherapy and the warfarin plus tigecycline combination therapy.

Warfarin coadministration does not alter the PK profile of tigecycline. However, administration of the therapeutic regimen of tigecycline increased the exposure (AUC) of R-warfarin and S-warfarin by 68% and 29%, respectively, but this increase in AUCs does not significantly alter warfarin's anticoagulant profile (measured by INR). No safety-related concerns are identified in healthy subjects after the administration of a single 25-mg oral dose of warfarin alone, multiple 50-mg IV doses of tigecycline, and the combined multiple-dose administration of 50 mg IV tigecycline and a single 25-mg oral dose of warfarin. Therefore, no dosage adjustment is warranted with coadministration of tigecycline and warfarin. However, consistent with good medical practice, the proper level of anticoagulant activity should be monitored whenever patient treatment is altered, such as initiating treatment with another drug.

This study only evaluates the effect of a single dose of warfarin on the pharmacokinetic profile of tigecycline. However, given tigecycline's main elimination pathways of biliary excretion of unchanged tigecycline, renal excretion of unchanged tigecycline, and glucuronidation, repeat-dose administration of warfarin would not be expected to produce a larger effect on the systemic clearance of tigecycline.

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

What is claimed is:

1. A composition comprising at least one glycylcycline chosen from a compound of formula 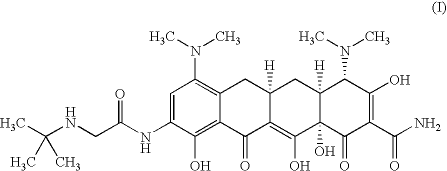
and pharmaceutically acceptable salts thereof, and at least one warfarin.

2. A composition according to claim 1, wherein the glycylcycline is a free base.

3. A composition according to claim 1, wherein the warfarin is warfarin sodium.

4. A composition according to claim 1, wherein the composition is suitable for parenteral administration.

5. A composition according to claim 1, wherein the composition is suitable for intravenous administration.

6. A pharmaceutical composition as claimed in claim 1 further comprising at least one pharmaceutically acceptable excipient.

7. A combination therapy comprising administration of at least one glycylcycline chosen from a compound of formula I 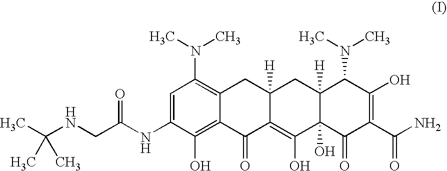
and pharmaceutically acceptable salts thereof, and at least one warfarin, wherein administration is simultaneous, separate or sequential

8. A combination therapy according to claim 7, wherein the warfarin dose may remain substantially the same as the dose before said combination therapy.

9. A combination therapy according to claim 7, wherein the glycylcycline and the warfarin are administered between twelve hours before and twelve hours after the at least one warfarin is administered.

10. A medical apparatus comprising at least two separate compartments, wherein a first compartment comprises at least one glycylcycline chosen from a compound of formula I 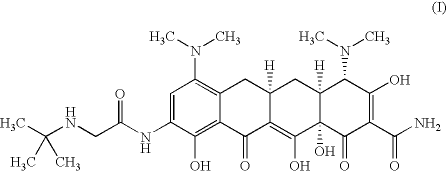
and pharmaceutically acceptable salts thereof and a second compartment comprising at least one warfarin, and wherein the first and second compartments are connected to at least one administration set.

11. A medical apparatus according to claim 10, wherein the first and second compartments are connected to the same administration set and are mixed prior to administration.

12. A medical apparatus according to claim 11, wherein the first and second compartments are connected to the same administration set and are mixed at a Y-site prior to administration.

13. A medical apparatus according to claim 10, wherein the warfarin dose may remain substantially the same as the dose before administration with at least one glycylcycline.

14. A method for administering at least one glycylcycline and at least one warfarin, comprising administering to a patient in need thereof a therapeutically effective amount of the at least one glycylcycline chosen from a compound of formula I 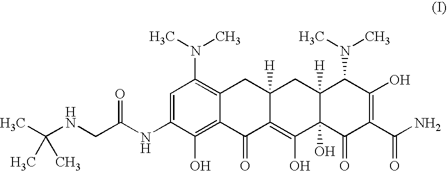
and its pharmaceutically acceptable salts, and administering to a patient in need thereof a therapeutically effective amount of the at least one warfarin.

15. A method of treating complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) comprising administering to a patient in need thereof a therapeutically effective amount of at least one glycylcycline chosen from a compound of formula I 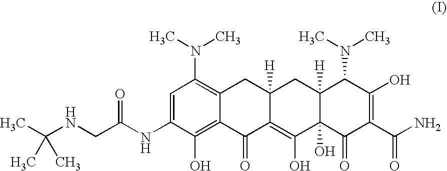
and its pharmaceutically acceptable salts, and administering to a patient in need thereof a therapeutically effective amount of at least one warfarin.

16. A method of treating complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof, which comprises providing to said patient an effective amount of a combination comprising a glycylcycline of formula I or a pharmaceutically acceptable salt thereof and a warfarin or pharmaceutically acceptable salt thereof.

17. A method of administering an antibiotic comprising administering to a patient in need thereof a therapeutically effective amount of at least one glycylcycline chosen from a compound of formula I 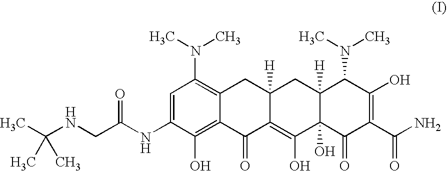
and its pharmaceutically acceptable salts, and administering to a patient in need thereof a therapeutically effective amount of at least one warfarin.

18. An article of manufacture, comprising (a) a dosage form of at least one glycylcycline chosen from a compound of formula I 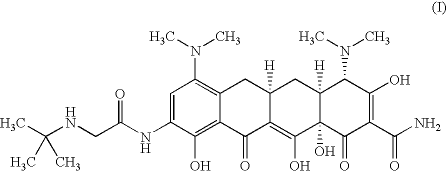
and its pharmaceutically acceptable salts,
(b) a package insert or printed labeling providing that a dosage form of the glycylcycline may be co-administered with warfarin wherein the warfarin dose may remain substantially the same as the dose before administration with the glycylcycline; and
(c) at least one compartment in which the glycylcycline is stored.

19. A kit comprising: (a) at least one dosage form of at least one glycylcycline chosen from a compound of formula I 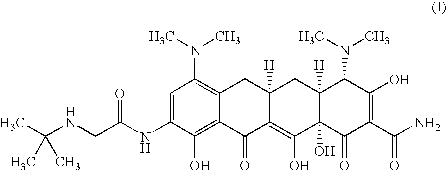
and its pharmaceutically acceptable salts; (b) at least one compartment in which the is stored; and (c) a package insert comprising: i) information regarding the dosage amount and duration of exposure of a dosage form of the glycylcycline and ii) providing that the dosage form of the glycylcycline may be administered with warfarin wherein the warfarin dose may remain substantially the same as the dose before administration with the glycylcycline.

20. Use of a glycylcycline of formula I or a pharmaceutically acceptable salt thereof in the preparation of a medicament for the treatment of complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof, which treatment also comprises administration of a warfarin or a pharmaceutical salt thereof.

21. Use of a warfarin or a pharmaceutically acceptable salt thereof in the preparation of a medicament for the treatment of complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof, which treatment also comprises administration of a glycylcycline of formula I or a pharmaceutical salt thereof.

22. A product comprising a glycylcycline of formula I or a pharmaceutically acceptable salt thereof and a warfarin or a pharmaceutically acceptable salt therfore as a combined preparation for simultaneous, separate or sequential use in the treatment of complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof.
